(19) United States
(12) Patent Application Publication
(10) Pub. No.: US 2004/0161764 A1
(43) Pub. Date: Aug. 19, 2004

(54) Preparation of calibrants and use thereof in the quantification of nucleotide sequences of interest

(76) Inventors: Jean Gabert, Callas (FR); Emmanuel Beillard, Venelles (FR)

Correspondence Address: IP DEPARTMENT OF PIPER RUDNICK LLP, ONE LIBERTY PLACE, SUITE 4900, 1650 MARKET ST, PHILADELPHIA, PA, 19103, US

(21) Appl. No.: 10/472,055

(22) PCT Filed: Mar. 15, 2002
(86) PCT No.: PCT/FR02/00937

(30) Foreign Application Priority Data
Mar. 15, 2001 (FR) 01/03551

(51) Int. Cl. C12Q 1/68; C07H 21/04

(52) U.S. Cl. 435/6; 435/320.1; 536/23.2

(57) ABSTRACT

The invention relates to the preparation of calibrants and the use thereof in the quantification of nucleotide sequences of interest.

The present invention relates to the preparation of calibrants and their uses in the quantification of nucleotide sequences of interest.

The quantification of nucleotide sequences of interest can be carried out according to several approaches:

by determination of the minimum sensitivity of an experiment (qualitative PCR)

by use of a competitor (competitive PCR), or,

by quantitative PCR in real time.

Qualitative PCR is concerned with the search for nucleotide sequences. It is used to establish the presence or absence of these sequences (final point technique). Consequently, no precise quantification is possible.

Competitive PCR is a quantitative PCR using a plasmid chimeric competitor. This technique assumes that the efficiency of the PCR is identical for the competitor, and the sought transcript, which are of different sizes. On the other hand, this technique uses a nested PCR which hugely increases the risk of contamination.

Quantitative PCR in real time aims to quantify via fluorescent molecules the nucleic acids of a sample. This is a real time technique, not a final point technique. The measurement is thus carried out during the exponential phase of the PCR, which makes it possible to assume a PCR yield of 100%. In the field of oncology, Marcucci et al. [1] were the first to describe this method. However, problems of stability were noted during the implementation of the method by the inventors. Moreover, the amplification yield of the sequence to be assayed of the calibrant does not reach 100% and the average expression of the ubiquitous gene used for the standardization of the assay cannot be correctly quantified. These difficulties then result in non-reproducibility of the assays.

Another problem arising in the gene quantification results from the consequent variations observed for the same assay carried out by different laboratories. The results obtained then appear dependent on the operator, the assay and the conditions in which the latter operates.

The inventors' work has led them to develop a method for quantification of nucleotide sequences of interest, in order to free themselves from all the external parameters which can lead to fluctuations in the results of an assay, and thus to obtain perfect reproducibility of the results of this assay, irrespective of the laboratory where this assay was carried out.

The invention thus provides a calibrant comprising a nucleotide sequence of interest to be assayed, inserted into a vector, characterized in that said calibrant is linear.

The term “calibrant” refers to a construction making it possible to carry out a quantitative assay.

The linearity of the calibrant is the essential parameter guaranteeing reproducibility. In fact, the linearity of the calibrant makes it possible to considerably improve the PCR yield.

The invention also proposes a method for preparing a calibrant comprising a vector, characterized in that it comprises the following stages:

insertion of a sequence of interest into the vector, and

linearization of the calibrant by a restriction enzyme selected such that said enzyme recognizes only one cleavage site on the vector, outside the insert.

Appropriate vectors are constituted by cloning vectors.

The calibrants which can be obtained by this preparation method fall within the scope of the invention.

Preferably, the calibrant is entirely artificial.

The manufacturing protocol of an entirely artificial calibrant is described in detail in Example 1.

This characteristic makes it possible to avoid any interference in the assay which could be due to the complexity of the biological material.

Moreover, the sequence of interest is preferentially DNA.

In fact, the use of DNA allows better stability over time of the calibrant according to the invention.

The vector used for the preparation of the calibrant according to the invention can advantageously be a plasmid.

The invention also relates to kits for carrying out the assays of nucleotide sequences of interest, characterized in that they comprise at least one dilution series of a calibrant according to the invention.

Advantageously, the kit can comprise two dilution series of two calibrants according to the invention, the first calibrant comprising a sequence to be assayed, the second calibrant comprising a sequence of a ubiquitous gene or gene transcript.

The second dilution series makes it possible to carry out a correct quantification of the ubiquitous gene or gene transcript in order to standardize the first assay.

In particular, the similarity of the operating conditions of the two assays makes possible the precision of the standardization.

According to a preferred embodiment, the dilution medium of the kit according to the invention is a buffer solution.

The choice of the buffer solution makes it possible to encourage the stability of the calibrants during their storage and during the assays.

Advantageously, the dilution medium moreover comprises nucleic acids having no homology with the sequences of interest of the calibrants.

The presence of nucleic acids in the dilution series mediums makes it possible to artificially reproduce the biological medium of the sample to be assayed. This in fact means carrying out all the assays under similar conditions in order to obtain the best reproducibility.

Preferably, the dilution medium is a buffer solution with a pH greater than or equal to 7.

This pH makes it possible to guarantee optimum stability of the calibrants.

According to a preferred embodiment, the nucleic acid introduced into the dilution media is the RNA of E. coli.

Preferably, the ubiquitous gene or gene transcript is chosen from the group made up of Abelson (ABL), β-glucuronidase (GUS) and β-2microglobulin (B2M).

These ubiquitous gene transcripts have been more particularly studied by the inventors to the extent that they are perfectly characterized (see Table 1 of Example 2). Their use thus makes it possible to increase reproducibility from one assay to another.

The kits according to the invention are particularly suitable for the assay of genes of interest such as AML1-ETO, E2A-PBX1, BCR-ABL, MLL-AF4, TEL-AML1, SIL-TAL, PML-RARA, CBFb-MYH11. Therefore, advantageously, the kits according to the invention can comprise dilution series of said genes of interest.

The sequences and the reference values of these fusion transcripts are described in Example 4.

According to a preferred embodiment, the calibrant series of the kits according to the invention are packaged in liquid or lyophilized form.

The invention also covers a method for preparing a kit for assaying a nucleotide sequence of interest, characterized in that it comprises the following stages:

preparation of a calibrant according to the method of claim 2, and,

production of a dilution series in a medium with a pH greater than or equal to 7, comprising the RNA of E. coli.

The calibrants according to the invention make it possible to carry out, with precision and reproducibility, the quantification of all kinds of nucleic acid in a sample to be analyzed, advantageously of nucleic acids associated with pathologies.

More precisely, the calibrants according to the invention are particularly suitable for the quantification of genes, of fusion transcripts and more particularly to the quantification of fusion transcripts involved in leukaemia.

The invention also provides a method for quantification of a target nucleic acid in a sample, characterized in that it comprises the following stages:

assay by quantitative PCR and establishment of a calibration curve using the dilution series of the calibrant carrying the target nucleotide sequence,

assay by quantitative PCR and establishment of a calibration curve using the dilution series of the calibrant carrying the ubiquitous gene or gene transcript sequence,

assay by quantitative PCR of the target sequence and ubiquitous gene or gene transcript sequence in the sample, using calibration curves and,

calculation of the standardized number of copies using the following formula:

Log(SNC)=Log TF−Log UG

Where SNC is the standardized number of copies,

TF the number of copies of the target sequence, and

UG the number of copies of the ubiquitous gene or transcript,

the assays preferably being duplicated.

This method is described in full detail in Example 2. Moreover, the assay carried out in Example 3 makes it possible to show an implementation of this method.

### EXAMPLE 1

### Preparation of a Calibrant

I. Brief Presentation of the Different Stages of the Operating Protocol.

A fusion transcript (MLL-AF4 for example) is amplified with the corresponding primers [2] starting with the cDNA originating from a cell line or from a patient. The PCR product is inserted into an adapted vector (pCR II TOPO plasmid for example) then cloned in a bacterial strain of E. coli competent for transformation.

The plasmid vector is then extracted according to the standard methods [3] and quantified by UV spectrophotometry.

This vector is then linearized. The restriction enzyme selected for the linearization should have only one cleavage site on the vector, outside the insert.

Given the size of the vector and the insert, the size of the plasmid construction is calculated (in base pairs). Its molecular weight is estimated for an average weight of nucleotides. The number of copies present in a given weight of digested plasmid is then determined.

A dilution series is then produced.

The plasmid standard is then ready for use.

II. Detailed Description of the Protocol.

A. Preparation of the DNA Sequences to be Inserted in a Cloning Vector

The preparation of the nucleotide sequences of interest before cloning in a vector is carried out:

either starting with an RNA transformed to cDNA by a reverse transcription (RT) stage optionally followed by the synthesis of a second strand of DNA,

or directly starting with a DNA.

The fragments of DNA obtained by one or other of the methods can then be amplified by PCR.

1. Preparation of DNA Sequences from RNA

The target DNA sequences are first prepared by reverse transcription (RT) of a total, ribosomal or messenger RNA of human, animal or vegetable origin, from pro- or eukaryotic, uni- or pluricellular organisms, or viruses.

The RT takes place according to RT Protocol. 1 [2] or Protocol 2 applied to long fragments.

Protocol 1: RT BIOMED-1

This protocol is based on Reference [2].

The reverse transcription requires primers.

They can be random (N(6)) or oligodT(30), optionally comprising an additional sequence making it possible to carry out an anchored PCR (for example, SEQ ID No.1: 5′ CGT CGT CGT GAA TTC CTA GAT CTT CTA GAT ATG TT 3′). The primers can optionally be modified at their ends for subsequent use.

The reverse transcription is carried out in two phases:

Denaturation of the RNA:

1 μg of RNA in 8.5 μl of water is denatured according to the following cycle: 10′ at 70° C., then maintenance at 20° C.

Synthesis of the cDNA:

The denatured RNA is mixed with 10.5 μl of the following reaction mixture: Buffer 5×(4 μl), dNTPNa—10 mM (2 μl), DTT—100 mM (2 μl), RT primer—10 μM (1 μl), RNase-inh—40U/μl (0.5 μl), reverse transcriptase—50U/μl (2 μl) The synthesis of the cDNA is carried out according to the following cycle: 10′ at 20° C.—45′ at 42° C.—3′ at 99° C.—4° C. infinite. This synthesis is optionally carried out with modified nucleotides.

Protocol 2: RT Applied to Long Fragments

The reverse transcription requires reagents which make it possible to form large cDNAs. ROCHE's Expand Reverse Transcriptase® kit is used for these experiments. The primers used are random (N(6)) or oligodT(30) primers optionally comprising an additional sequence making it possible to carry out an anchored PCR such as SEQ ID No.1 described above. The primers can optionally be modified at their ends for subsequent use.

The reverse transcription is carried out in two phases:

Denaturation of the RNA:

1 μg of RNA in 8.5 μl of water is denatured according to the following cycle: 10′ at 70° C., then maintenance at 20° C.

Synthesis of the cDNA:

The denatured RNA is mixed with 10.5 μl of the following reaction mixture: Buffer 5×(4 μl), dNTPNa—10 mM (2 μl), DTT—100 mM (2 μl), RT primer—10 μM (1 μl), RNase-inh—40U/μl (0.5 μl), Expand RT—50U/μl (1 μl). The synthesis of the cDNA is carried out according to the following cycle: 10′ at 20° C.—45′ to 2 hours at 42° C.—3′ at 99° C.—4° C. infinite. This synthesis can be carried out with modified nucleotides.

Treatment with RNase H:

2 μl of RNase H (50 U/μl) are added to 20 μl of the previous mixture, and incubated for 10′ at 42° C., then the tube is cooled down to 20° C. The whole mixture is then diluted to ⅓, final volume 60 μl.

2. DNA Sequences

The DNA sequences are obtained either directly from DNA, or by reverse transcription from RNA.

If the target sequences are obtained directly from DNA, the latter can be of human, animal or vegetable origin, from pro- or eukaryotic, uni- or pluricellular organisms, viruses, from coding or non-coding, intronic or exonic sequences.

If the target sequences are obtained from RNA, the latter is treated according to the previous paragraph in order to obtain a first strand of complementary DNA.

A second strand of DNA can be synthesized from the sequence obtained by RT after addition of a 3′ tail (tailing), by operating, for example, according to protocol 3 or ligation with a predefined sequence optionally containing a site recognized by a restriction enzyme at its end.

Protocol 3: Tailing for the Synthesis of a Second Strand.

The tailing is carried out on the product prepared by protocol 2 and based on reference [3].

Purification of the cDNA:

The purification is carried out on a Sephadex G50® column. The cDNA is then eluted in 50 μl of water or Tris EDTA 10 mM/1 mM.

The tailing is carried out as follows:

In a volume of 24 μl: Sterile water (9.5 μl), Buffer (10×) 1.25 μl, optionally modified dCTP [2 mM] 2.5 μl, MgCl₂ [50 mM] (0.75 μl), purified cDNA (10 μl).

Incubate for 2 to 3 minutes at 94° C. Cool down for 1 minute in ice.

Add the TdT, homogenize and incubate for 15 minutes at 37° C.

Inactivate the TdT for 10 minutes at 70° C.

Once the DNA is obtained (directly or indirectly, single-strand or not single-strand, the sequences can optionally be amplified by PCR or directly inserted in a vector.

The PCR is carried out starting with a pair of known primers (reference 1) or primers defined according to the invention (optionally comprising at their 5′ end a sequence recognized by a restriction enzyme) making it possible to amplify the target according to protocol 4 of PCR BIOMED-1 or protocol 5 of PCR of long fragments.

Protocol 4: PCR BIOMED-1

This PCR protocol is based on reference [2].

It makes it possible to amplify the sequences of interest targeted by the primers (A and B) of reference [2], and in particular the fusion genes of reference [2] or ubiquitous genes. These primers can optionally be modified at their ends for subsequent use.

The amplification cycle is as follows: 94° C.—3′, 35×(94° C.—30″, 65° C.—30″, 72° C.—1′), 16° C.—infinite. It is carried out in the presence of Taq DNA polymeraseo, optionally modified nucleotides and 10 μl of RT product or 1 μl of DNA. The fragments obtained are short in size and can then be cloned in suitable vectors.

Protocol 5: long fragment PCR(standard, anchored or bi-anchored)

The amplification programme and the Taq DNA polymerase®, make it possible to obtain fragments of at least Skb with specific primers. The amplification is carried out with dNTPs optionally replaced by modified nucleotides.

The amplification cycle is as follows:

94° C.—0″, 10×(94° C.—10″, 68° C.—8′), 20×(94° C.—10′, 68° C.—8′+20″ per cycle), 68° C.—7′, 16° C.—infinite).

In a 50 μl PCR well, the reaction mixture is as follows:

RT or PCR product (10 μl), H₂O DEPC (21 μl), dNTP—10 mM (2.5 μl), Sense or anchor primer corresponding to SEQ ID No. 1 above 100 μM (0.5 μl), Antisense primer or anchor 100 μM (0.5 μl), PCR buffer (5 μl), MgCl₂ at 3 mM final, Taq DNA polymerase® 3.5U. The anchor corresponds to any defined sequence, the sequence given here is an example comprising cleavage sites for restriction enzymes.

This amplification can optionally be followed by a second one, with internal primers in order to increase the specificity of the amplification. The primers can optionally be modified at their ends for subsequent use.

3. Purification of the PCR Products

For the purposes of purification, the products are first arranged on gel in order to check the size and relative quantity with respect to a molecular weight marker of known concentration. The quantity of the product is estimated with respect to the molecular weight marker. In the presence of several bands, isolation of the band of interest can be carried out by the cutting out on the gel of the sought band and extraction of the DNA of interest.

These products are then optionally purified by precipitation from ethanol, then taken up in a volume of Tris EDTA 10 mM/1 mM in order to be assayed by UV spectrophotometry [3] or by spectrofluorimetry using an intercalating fluorescent agent.

B. Cloning of the Sequences

The products prepared are then cloned. To this end, they are inserted in a cloning vector, then transferred into a host organism of human, animal, viral or vegetable origin, pro- or eukaryotic, uni- or pluricellular organisms, in order to produce the sequence of interest in large quantity.

1. Production of the Sequence in prokaryotic or Viral Organisms

The sequence(s) of interest is (are) ligated to the vector by a ligase or topoisomerase at the cloning site. The plasmid vector (Bluescript® or equivalent), phage vector (Lambda or equivalent), cosmid, viral or also a YAC (yeasts) or BAC vector containing the insert is amplified in large quantity in order to be extracted.

The receiving host cell is advantageously an Escherichia coli bacterial strain, rendered competent for transformation by a physical or chemical method. The clone (vector+insert) can be introduced into other bacterial strains or prokaryotic organisms, for example yeasts or insect cells. The extraction of the vector carrying the insert is carried out according to standard techniques [3].

2. Production of the Sequence in Eukaryotic Organisms

The sequence(s) of interest is (are) inserted into eukaryotic vectors by a ligase or topoisomerase at the cloning site, then transferred into unicellular eukaryotiq organisms (for example, Saccharomyces sp or Candida sp), pluricellular organisms or into human, animal or vegetable immortalized lines.

3. Qualitative Control of the Insert and Sequencing

The selection of the organism carrying the vector is advantageously carried out according to the usual methods [3] and the presence of the insert is verified by PCR by choosing non-specific primers situated on the vector or primers specific to the insert by operating in particular according to protocol 4 in the examples.

The sequencing of the product inserted is carried out in order to determine the presence of any mutations in the cloned product and the sense of insertion of the product. The sequencing is carried out according to the standard technologies starting with universal primers situated on the cloning vector (M13, Sp6 or T7 for example) or starting with primers specific to the insert.

C. Quantification of the Gene of Interest

The calibrant can comprise the gene of interest (insert) in different forms: double or single-stranded DNA, RNA.

1. Double-Stranded DNA

The cloning vector is extracted from the cells by standard extraction techniques (SAMBROOK). The product is taken up in a solution of Tris EDTA 10 mM/1 mM pH=8, quantified by UV spectophotometry [3] or by spectrofluorimetry using an intercalating agent.

Then 20 μg of this product are digested in 20 μl for one hour at 37° C. with 10 U of a suitable restriction-enzyme (BamHI, HindIII, or another) in order to linearize the vector.

The number of copies of the sequence of interest or its equivalent in mass units is then determined, taking account of the molecular weight of the vector, the size of the insert and the quantity of the product cloned in a determined volume (Formula 1)

MW_{c}=(NTV+NTI)*MW_{N}/N Formula 1

where

MW_{c} is the molecular weight (g) of a vector copy carrying 1 insert,

NTV, the number of nucleotides in the vector,

NTI, the number of nucleotides in the insert,

MW_{N}, the average molecular weight of a nucleotide, i.e. 321.44 g, and,

N, Avogadro's number, i.e. 6.02 10²³.

2. Single-Stranded DNA

From the linearized vector (see previous paragraph), carrying the universal sequences M13F and M13R, a single-stranded DNA sequence is produced using a polymerase (KLENOW type or equivalent) in the presence of the corresponding primer in order to obtain the desired strand, according to standard protocols (SAMBROOK). The product is then purified on gel then taken up in a solution of Tris EDTA 10 mM/1 mM, pH=8, quantified by UV spectrophotometry [3] or by spectrofluorimetry using an intercalating agent.

The number of copies of the sequence of interest or its equivalent in mass units is then determined, taking account of the size of the insert and the quantity of the product in a determined volume (Formula 1).

3. RNA

In order to prepare from RNA, advantage is taken of the fact that the cloning vector (pCR II TOPO or Blue Script) carrying the sequence of interest described above possesses the promoters for the RNA polymerases SP6 or T7. These polymerases are used for the production of single-stranded RNA from the promoter sites on the vector according to the usual protocols [3]. The choice of the polymerase is a function of the sense of insertion of the sequence of interest.

The RNA produced is then purified according to standard isolation techniques [3]. It is then assayed by UV spectrophotometry [3] or by spectrofluorimetry using an intercalating agent (Hoescht or Interchim).

Finally, the number of copies of the sequence of interest or its equivalent in mass units is determined taking account of the molecular weight of the insert and the quantity of the product cloned in a determined volume (Formula 1).

D. Dilution of the Preparations

The sequences of interest are then diluted so that their exact quantity in a determined volume is known.

The final product can be a sample or a series of dilutions in tandem of the sample comprising one or more sequences of single- or double-stranded RNA and/or DNA, carried by the same fragment of nucleic acid or by different fragments.

The quantity can be expressed in the form of mass units of the reference sequence or as an absolute or relative number with respect to a second sequence also present in a known quantity in the preparation. By extension, a mixture of a large number of sequences of known quantity in the same sample can be prepared in the same manner.

The final product can be lyophilized or in solution in a suitable solvent, optionally comprising inert or active agents.

1. Practical Production of DNA Dilutions

In order to produce dilutions of DNA from a known quantity of single- or double-stranded RNA, a dilution in tandem of 10 to 10 or half to half of this sample is carried out. These dilutions are carried out in an RNA (dilutions carried out in an RNA of human, animal or vegetable origin, from pro- or eukaryotic, uni- or pluricellular organisms, or viruses) or a DNA in solution in a Tris EDTA 10 mM/1 mM buffer, pH=8, in the presence of one or more inert substances (glycerol, bovine serum albumin, powdered milk, Dextran, eukaryotic ribosomal RNA 16 and 23S, or any other chemical stabilizing product.

2. Practical Production of RNA Dilutions

In order to produce dilutions of RNA, a known quantity of RNA is diluted in tandem from 10 to 10 or from half to half. These dilutions are carried out in an RNA (of human, animal or vegetable origin, from pro- or eukaryotic, uni- or pluricellular organisms, or viruses) or a DNA in solution in a Tris EDTA 10 mM/1 mM, pH=8, in the presence of one or more inert substances (glycerol, bovine serum albumin, powdered milk, Dextran, or any other chemical stabilizing product.

For example, the first solution, referred to as 10-1 will comprise 2.10⁹ copies of the plasmid per microlitre of solution, obtained from a volume V of the enzyme digestion diluted in a final volume of 500 μl of DEPC water. Dilutions in tandem are then carried out in a Tris EDTA 10 mM/1 mM buffer, pH=8, containing 20 ng/μl of 16S and 23S RNA of E. coli in order to obtain final concentrations of 200,000, 20,000, 2,000, 200, 20 and 2 copies per microlitre of solution.

The preparations are then checked.

E. Checking the Preparations

1. Qualitative Control of the Presence of the Insert

The qualitative controls of the presence of the insert are carried out in order to determine the presence of the sought insert in the cloned product. The appearance of the band(s) amplified on gel make it possible to assess the quality of the preparation. This control is carried out by PCR starting with universal primers situated on the cloning vector or with primers which have served to produce the PCR fragment according to the BIOMED-1 amplification programme. It is carried out directly from DNA samples or after RT (in particular according to protocol 1 or 2 from RNA samples).

2. Quantitative Control of the Presence of the Insert

The quantitative controls are carried out in order to determine the quantity of sequence of interest or vector. This control can be carried out by PCR starting with universal primers situated on the cloning vector or with primers which have served to produce the PCR fragment according to the amplification programme of protocol 6.

Protocol 6: Taqman®-Type Quantitative PCR

The amplification cycle is as follows:

50° C.—2′, 94° C.—2′, 50×(94° C.—3″, 60° C.—1′) 16° C.—infinite.

The composition of the reaction mixture is as follows:

DNA or cDNA sample (100 ng of DNA or its equivalent)

Sense and antisense primers, 300 nM final

Taqman® probe labelled with fluorophores (200 nM final) or its equivalent (fluorescent intercalating agent, SYBR green© for example) allowing the revelation of the amplified product or reaction mixture containing these components.

Reaction mixture containing the PCR buffer, the polymerase, and any other component allowing quantification.

The control is carried out directly starting with DNA samples or after RT starting with RNA samples (in particular according to protocol 1 or 2). The quantification can also be carried out by current techniques for measuring the nucleotide sequences, in solution or on solid supports by means of substances adapted to quantification.

It will be observed that, except for the products obtained from a direct dilution of the vector (double-stranded DNA) in the buffer solution, the vector sequences do not occur at any time in the final product.

### EXAMPLE 2

### Quantification Method

The quantification comprises two principle stages:

the first stage consists of assaying the gene of interest,

the second stage aims to standardize the assay carried out.

The standardization is carried out by the assay of a gene referred to as “ubiquitous”.

All the assays are carried out by quantitative PCR.

For each stage, a calibration curve is established, using the calibrant according to the invention, referred to as a “plasmid standard curve”. The assay of the gene of interest and of the ubiquitous gene, present in the sample, is then carried out. The assay values are then determined by interpolation, on the calibration curve.

A. Quantification of the Ubiquitous Gene or Gene Transcript in the Sample

By “ubiquitous gene” is meant a reference gene serving to standardize the experiments. Its quantification is precise and reproducible from one experiment to another.

It can also be the ubiquitous gene transcript. There are a number of ubiquitous genes (β-globin, β-actin, GAPDH or transcripts of genes such as Abelson, β-2 microglobulin or B2M, β-glucoronidase or GUS, Porphobilinogen deaminase or PBGD, Tata Box binding protein or TPB, etc.) the expression of which is approximately known in the cells of the human body.

By way of example, the preferred ubiquitous genes as well as their experimental values, are described in the following table:

TABLE 1

| | N | average 10^{NC} | Range |
|---|---|---|---|
| ABL | | | |
| All samples | 316 | 18071 | 1345-127643 |
| Leukaemic sample | 190 | 13378 | 1175-151356 |
| “Normal” samples | 126 | 19953 | 1380-53703 |
| B2M | | | |
| Leukaemic sample | 190 | 781628 | 9977-5370317 |
| “Normal” PB and PBSC | 100 | 1445440 | 208929-6760829 |
| “Normal” bone marrow | 26 | 1071519 | 28641-1927524 |
| GUS | | | |
| Leukaemic sample | 190 | 40926 | 2944-344349 |
| “Normal” PB and PBSC | 52 | 66069 | 7177-195434 |
| “Normal” bone marrow | 74 | 21878 | 2630-70794 |

N: number of samples
Average 10^{NC}: average number of copies in a sample per 10 ng of total RNA originating from this sample.
Range: value above and below the range comprising 95% of the samples.
PB: Peripheral blood
PBSC: Peripheral blood strain cells

The ubiquitous gene is chosen as a function of various parameters:

firstly, its expression must be stable under experimental conditions,

its expression must also be correlated with that of the target during the degradation process, i.e. its expression remains in a constant relation to that of the target.

The ubiquitous gene is prepared according to the same method as the gene of interest. Thus a dilution series is obtained of calibrants containing the ubiquitous gene.

The assay of the “ubiquitous gene” or its transcript will provide information on the quality of the assay carried out. In fact, the assay is carried out by quantitative PCR which can translate a degradation of the assayed DNA or RNA, in particular in the sample which represents a complex medium. It is assumed that this degradation of the assayed DNA or RNA will be accompanied by a similar degradation, in the same proportions, of the “ubiquitous gene or transcript”. If the exact quantity of ubiquitous gene determined by the PCR is known, the degradation of said gene can then be deduced from the latter and, according to the postulate set forth above, that of the gene of interest to be assayed in the sample.

A plasmid standard curve is established over 3 logarithmic dilutions assayed in duplicate in the same experiment. The position of the first and second point of dilution is determined with respect to the average expression observed of the gene studied. The number of copies present in the sample considered is determined by interpolation.

For example, the average expression of the ABL ubiquitous gene is 20,000 copies per 0.1 μg of RNA equivalent. The first plasmid dilution is calculated in order to contain 100,000 copies in 5 μl of solution. The second and third will contain 10,000 and 100 copies respectively in 5 μl of solution. In this fashion, any degradation in the RNA of the sample is translated by a number of weaker copies and will be contained in the second and third dilutions.

B. Quantification of the Sequence of Interest

A plasmid standard curve is established over 4 to 5 logarithmic dilutions assayed in duplicate in the same experiment. The first range point corresponds to 1,000,000 copies of the gene and the last to 10 copies. The intermediate dilutions are 100,000, 1,000 and (or) 100 copies. The number of copies present in the sample considered is determined by interpolation.

C. Expression of the Results

The amplification of the PCR products follows a geometric law. The statistical distribution of the number of copies in a sample for a repeated assay is logarithmic. It is therefore necessary to consider the logarithmic values in order to carry out the standardization calculations.

Thus, for a sample assayed in duplicate for a given gene of interest, for example, a fusion transcript (FT1 and FT2) and its ubiguitous gene (UG1 and UG2), the value obtained follows the following expression:

Log (SNC)=(Log FT1+Log FT2)/2−(Log UG1+Log UG2)/2+NC Formula 2

Where SNC is the standardized number of copies

FT, the number of copies of the target sequence (for example, fusion transcript),

UG, the number of copies of the ubiquitous gene or gene transcript,

NC (optional) is the logarithm of the median number of copies observed for the gene considered (e.g.: for ABL, NC=4). This makes it possible to express the result for 10,000 copies of ABL.

The term “Log” refers to a decimal logarithm.

The results can then be related to derived units: to the number of moles, grams or their derivatives.

### EXAMPLE 3

### Assay of a Transcript of a Target Related to a Reference Trancript

The amplification of a fusion transcript .(MLL-AF4) and a ubiquitous gene (ABL) is carried out in parallel by quantitative PCR. The raw machine results are shown in the following table:

TABLE 2

| | MLL-AF4 (Ct) | | ABL (Ct) | |
|---|---|---|---|---|
| 10^{NC} | Replicate 1 | Replicate 2 | Replicate 1 | Replicate 2 |
| Patient | 27.9 | 28.1 | 26.98 | 27.02 |
| Plasmid 10⁶ | 19.35 | 19.26 | | |
| copies | | | | |
| Plasmid 10⁵ | 22.84 | 22.85 | 23.46 | 23.55 |
| copies | | | | |
| Plasmid 10⁴ | | | 27.43 | 27.09 |
| copies | | | | |
| Plasmid 10³ | 30.42 | 30.59 | 30.3 | 30.5 |
| copies | | | | |
| Plasmid 10² | 34.74 | 37.79 | | |
| copies | | | | |
| Plasmid 10¹ | 37.17 | 40.18 | | |
| copies | | | | |

| | | |
|---|---|---|
| Regression | Ct = −3.88 NC + 42.4 | Ct = −3.43 NC + 40.8 |
| line | | |
| Patient | NC = 3.71, i.e. 5149 copies | NC = 4.01, i.e. 10272 copies |
| results | | |

Example of amplification in parallel of 2 ABL and MLL-AF4 transcripts on plasmids and a sample from a patient at diagnosis.

The raw values (Ct) of the plasmid dilutions given by the machine are used to construct the regression line by plotting the log of the number of copies along the x-axis and the Ct of the dilution along the y-axis. The corresponding regression lines are shown in Table 2. The number of corresponding copes is calculated by interpolation.

Using formula 2, the result obtained for the patient is calculated according to:

Log(SNC)=3.71−4.01=−0.30

where

SNC=10^{(-0.3)}=0.501 copies of MLL-AF4 per copy of the reference transcript (ABL).

### EXAMPLE 4

### Assay of Transcripts Involved in Leukaemia

1. Presentation of the Transcripts

The fusion transcripts result from the fusion of two genes.

The table below provides the accession numbers in the Genbank® database, of sequences of the two genes having fused in order to produce the transcript described.

TABLE 3

| Fusion transcript | Gene 1 accession No. | Gene 2 accession No. |
|---|---|---|
| AML1-ETO | D43369 | D14289 |
| E2A-PBX1 | M31222 | M86546 |
| BCR-ABL | X02596 | X16416 |
| MLL-AF4 | L04284 | L13773 |
| TEL-AML1 | U11732 | D43369 |
| SIL-TAL | M74558 | S53245 |
| PML-RARA | M73778 | X06538 |
| CBFb-MYH11 | L20298 | D10667 |

2. Determination of the Reference Values

The expression level values of the main fusion transcripts involved in leukaemia have never to date been presented in their entirety.

The following tables illustrate these values. They can be used as comparison values following an assay, being producible according to the quantification method according to the invention. These assays can in particular be intended for diagnosis (as part of a diagnostic method, for example) or in order to assess the effectiveness of a treatment concerning any pathological state for which diagnosis has demonstrated a gene anomaly (gene or transcript) which can be detected by the method proposed. In particular, the assay and diagnosis methods according to the invention allow the monitoring of anti-leukaemia treatments.

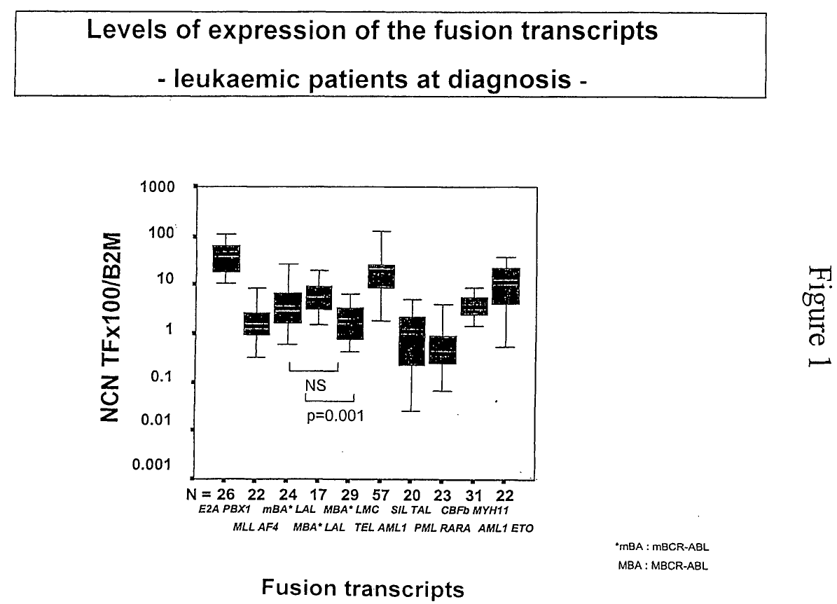

All these values are summarized in FIG. 1. In this figure, FT refers to the fusion transcripts, SNC to the standardized number of copies, ALL to an acute lymphoblastic leukaemia and CML to chronic myeloid leukaemia.

In the following tables, unless otherwise specified, the standardized number of copies of the fusion gene is given for a copy of the ABL gene, the GUS gene or 100 copies of B2M. The median value is defined for a range covering 95% of the values. The correlation coefficient (Correl. Coef.) is defined before standardized by the ubiquitous gene.

No statistically significant difference has been established between the results obtained from the blood and bone marrow samples.

TABLE 4
Amplification of the ABL, B2M, GUS and
E2A-PBX1 genes in the cell line and in
patients at diagnosis.

| | Standardized number of copies | | |
|---|---|---|---|
| | E2A-PBX1/ | E2A-PBX1 × 100 | E2A-PBX1/ |
| Samples | ABL | B2M | GUS |
| Cell line | | | |
| 697 | 5.0 | 33.9 | 9.0 |
| Patients | | | |
| Bone marrow | 7.6 | 44.6 | 8.9 |
| (n = 14) | [2.1-19.5] | [10.5-101.0] | [1.6-17.8] |
| Blood | 9.4 | 42.7 | 13.6 |
| (n = 13) | [3.8-72.4] | [2.2-115.9] | [2.5-67.6] |
| Correl. coef. | 0.83 | 0.62 | 0.66 |

TABLE 5
Amplification of the ABL, B2M, GUS and
MLL-AF4 genes in cell lines and in
patients at diagnosis.

| | Standardized number of copies | | |
|---|---|---|---|
| | MLL-AF4/ | MLL-AF4 × 100 | MLL-AF4/ |
| Samples | ABL | B2M | GUS |
| Cell lines | | | |
| RS4; 11 | 0.35 | 4.4 | 0.29 |
| MVA-11 | 0.56 | 9.3 | 0.63 |
| ALL-PO | 0.62 | 9.3 | 0.59 |
| Patients | | | |
| Bone marrow | 0.46 | 1.2 | 0.32 |
| (n = 14) | [0.09-0.98] | [0.07-8.3] | [0.04-0.65] |
| Blood | 0.60 | 1.4 | 0.27 |
| (n = 13) | [0.16-0.91] | [0.32-2.9] | [0.09-0.58] |
| Correl. coef. | 0.79 | 0.67 | 0.76 |

TABLE 6
Amplification of the ABL, B2M, GUS and
TEL-AML1 genes in the REH cell line and
in patients at diagnosis.

| | Standardized number of copies | | |
|---|---|---|---|
| | | TEL- | |
| | TEL-AML | AML1 × 100/ | TEL-AML1/ |
| Samples | 1/ABL | B2M | GUS* |
| REH | | | |
| Cell line | 3.2 | 27 | 2.5 |
| Patients | | | |
| Bone marrow | 4.45 | 23 | 3.19 |
| (n = 30) | [0.46-32.70] | [0.63-132] | [0.27-10.33] |
| Blood | 4.71 | 12 | 2.63 |
| (n = 13) | [0.20-15.2] | [0.39-99] | [0.50-9.00] |
| Correl. coef. | 0.66 | 0.42 | 0.66 |

*12 blood samples and 17 bone marrow samples were tested for GUS.

TABLE 7
Amplification of the ABL, B2M, GUS and
BCR-ABL(mBCR) genes in the cell line
and in patients at diagnosis.

| | Standardized number of copies | |
|---|---|---|
| | BCR- | BCR- |
| Samples | ABL(mBCR) × 100/B2M | ABL(mBCR) × 100/GUS |
| Patients | | |
| Bone marrow | 2.69 | 1.48 |
| (n = 17) | [0.58-26.3] | [0.24-16.6] |
| Blood | 3.16 | 1.62 |
| (n = 13) | [0.03-13.8] | [0.18-9.33] |

TABLE 8
Amplification of the ABL, B2M, GUS and
BCR-ABL(MBCR) genes in the cell line
and in patients at diagnosis.

| | Standardized number of copies | |
|---|---|---|
| | BCR-ABL(MBCR) × | BCR-ABL(MBCR)/ |
| Samples | 100/B2M | GUS |
| Cell line | | |
| K562 | 2.24 | 0.66 |
| Patients | | |
| Bone marrow | 0.95 | 1.12 |
| (n = 15) | [0.43-5.3] | [0.04-0.87] |
| Blood | 2.8 | 0.22 |
| (n = 14) | [0.56-6.2] | [0.08-0.41] |
| Correl. coef. | 0.63 | 0.76 |
| Patients | | |
| Bone marrow and Blood | 4.90 | 1.15 |
| (n = 17) | [0.14-19.5] | [0.03-3.89] |
| Correl. coef. | 0.56 | 0.60 |

TABLE 9
Amplification of the ABL, B2M, GUS and
SIL-TAL genes in the cell lines and
in patients at diagnosis.

| | Standardized number of copies | | |
|---|---|---|---|
| | SIL-TAL/ | SIL-TAL/ | SIL-TAL/ |
| Samples | ABL | 100B2M | GUS |
| Cell lines | | | |
| CEM | 0.12 | 1.38 | 0.07 |
| ALL1 | 0.11 | 0.79 | 0.09 |
| Molt-15 | 0.10 | 0.27 | 0.06 |
| Molt-16 | 0.08 | 0.45 | 0.11 |
| HSB2 | 0.41 | 3.47 | 0.44 |
| PF382 | 0.07 | 0.47 | 0.11 |
| Patients | | | |
| Bone marrow | 0.12 | 1.24 | 0.15 |
| (n = 10) | [0.02-0.23] | [0.03-4.86] | [0.02-0.44] |
| Blood | 0.09 | 0.86 | 0.12 |
| (n = 10) | [0.02-0.21] | [0.05-3.28] | [0.01-0.26] |

TABLE 10
Amplification of the ABL, B2M, GUS and
PML-RARA genes in patients at diagnosis.

| | Standardized number of copies | | |
|---|---|---|---|
| | PML-RARA/ | PML-RARA × 100/ | PML-RARA/ |
| Samples | ABL | B2M | GUS |
| Patients | | | |
| Bone marrow | 0.30 | 0.72 | 0.08 |
| (n = 14) | [0.09-1.82] | [0.19-3.18] | [0.02-0.56] |
| Blood | 0.36 | 0.26 | 0.05 |
| (n = 9) | [0.11-0.78] | [0.07-0.60] | [0.02-0.13] |
| Correl. coef. | 0.80 | 0.54 | 0.73 |

TABLE 11
Amplification of the ABL, B2M, GUS and
CBFb-MYH11 genes in the cell line and
in patients at diagnosis.

| | Standardized number of copies | | |
|---|---|---|---|
| | CBFb-MYH11/ | CBFb-MYH11 × 100/ | CBFb-MYH11/ |
| Samples | ABL | B2M | GUS |
| Cell line | | | |
| ME-1 | 0.90 | 4.64 | 0.40 |
| Patients | 1.35 | 3.98 | 0.42 |
| (n = 24) | [0.13-14.8] | [1.45-447] | [0.07-4.57] |
| Correl. coef. | 0.76 | 0.65 | 0.76 |

TABLE 12
Amplification of the ABL, B2M, GUS and
AML1-ETO genes in the cell line and
in patients at diagnosis.

| | Standardized number of copies | | |
|---|---|---|---|
| | AML1-ETO/ | AML1-ETO × 100/ | AML1-ETO/ |
| Samples | ABL | B2M | GUS |
| Cell line | | | |
| KASUMI-1 | 5.46 | 80 | 1.42 |
| Patients | | | |
| Bone marrow | 1.5 | 5.2 | 0.43 |
| (n = 8) | [0.26-3.0] | [0.26-11] | [0.054-1.5] |
| Blood | 2.1 | 7.8 | 0.48 |
| (n = 13) | [0.17-5.9] | [0.21-170] | [0.027-2.7] |
| Correl. coef. | 0.81 | 0.80 | 0.44 |

### REFERENCES

[1] Marcucci et al., “Detection of minimal residual disease in patients with AML1/ETO-associated acute myeloid leukemia using a novel quantitative reverse transcription polymerase chain reaction assay”, Leukemia, 1998, 12: 1482-1489.

[2] Van Dongen et al., Report of the European BIOMED 1 concerted Action, Leukemia, 1999.

[3] SAMBROOK et al., MOLECULAR CLONING a laboratory manual 2^{nd} Ed. Cold Spring Harbor Laboratory Press, 1989.

1. Calibrant comprising a nucleotide sequence of interest to be assayed, inserted into a vector, characterized in that said calibrant is linear.

2. Method for preparing a calibrant comprising a vector, characterized in that it comprises the following stages:
insertion of a sequence of interest into the vector, and
linearization of the calibrant by a restriction enzyme selected such that said enzyme recognizes only one cleavage site on the vector, outside the insert.

3. Calibrant which can be obtained by the method according to claim 2.

4. Calibrant according to claim 1 or 3, characterized in that it is entirely artificial.

5. Calibrant according to any one of claims 1, 3 and/or 4, characterized in that the sequence of interest is DNA.

6. Calibrant according to any one of claims 1, 3 to 5, characterized in that the vector is a plasmid.

7. Kit for assaying a nucleotide sequence of interest, characterized in that it comprises at least one dilution series of a calibrant according to any one of claims 1, 3 to 6.

8. Kit according to claim 7, characterized in that it comprises two dilution series of two calibrants according to any one of claims 1, 3 to 6, the first calibrant comprising a sequence to be assayed, the second calibrant comprising a ubiquitous gene or gene transcript sequence.

9. Kit according to claim 7 or 8, characterized in that the dilution medium is a buffer solution.

10. Kit according to any one of claims 7 to 9, characterized in that the dilution medium moreover comprises nucleic acids having no homology with the sequences of interest of the calibrants.

11. Kit according to claim 9, characterized in that the buffer solution has a pH greater than or equal to 7.

12. Kit according to claim 10, characterized in that the nucleic acid is RNA of E. coli.

13. Kit according to any one of claims 8 to 12, characterized in that the ubiquitous gene or gene transcript is chosen from the group composed of ABL, GUS and B2M.

14. Kit according to any one of claims 8 to 13, characterized in that the gene of interest is chosen from the group composed of AML1-ETO, E2A-PBX1, BCR-ABL, MLL-AF4, TEL-AML1, SIL-TAL, PML-RARA, CBFb-MYH11.

15. Kit according to any one of claims 8 to 14, characterized in that the series of calibrants are packaged in liquid or lyophilized form.

16. Method for preparing a kit for assaying a nucleotide sequence of interest, characterized in that it comprises the following stages:
preparation of a calibrant according to the method of claim 2, and,
production of a dilution series in a medium with a pH greater than or equal to 7, comprising RNA of E. coli.

17. Use of calibrants according to any one of claims 1, 3 to 6 and/or kits according to any one of claims 7 to 15, for the quantification of nucleic acids in a sample to be analyzed.

18. Use of calibrants according to claims 1, 3 to 6, and/or kits according to any one of claims 7 to 15, for the quantification of genes.

19. Use of calibrants according to claims 1, 3 to 6, and/or kits according to any one of claims 7 to 15, for the quantification of fusion transcripts.

20. Use of calibrants according to claims 1, 3 to 6, and/or kits according to any one of claims 7 to 15, for the quantification of fusion transcripts involved in leukaemia.

21. Method for the quantification of a target nucleic acid in a sample, characterized in that it comprises the following stages:
assay by quantitative PCR and establishment of a calibration curve using the dilution series of the calibrant carrying the target nucleotide sequence.
assay by quantitative PCR and establishment of a calibration curve using the dilution series of the calibrant carrying the sequence of the ubiquitous gene or gene transcript,
assay by quantitative PCR of the target sequence and of the ubiquitous gene or gene transcript in the sample, using calibration curves, and,
calculation of the standardized number of copies using the following formula:
Log(SNC)=Log TF−Log UG
Where SNC is the standardized number of copies,
TF the number of copies of the target sequence, and
UG the number of copies of the ubiquitous gene or transcript,
the assays preferably being duplicated.


SEQUENCE LISTING (2 sequences)

SEQ ID NO: 1 - DNA, 35 bp, Artificial Sequence
  Description of Artificial Sequence Primer
cgtcgtcgtgaattcctagatcttctagatatgtt

SEQ ID NO: 2 - DNA, 30 bp, Artificial Sequence
  Description of Artificial Sequence Synthetic oligonucleotide
tttttttttttttttttttttttttttttt